(19) United States
(12) Patent Application Publication
(10) Pub. No.: US 2026/0368984 A1
(43) Pub. Date: Nov. 19, 2026

(54) GENERATING PERSONALIZED THREE-DIMENSIONAL AVATARS FOR PATIENT REHABILITATION

(71) Applicant: International Business Machines Corporation, Armonk, NY (US)

(72) Inventors: Sarbajit Kumar RAKSHIT, Kolkata (IN); Natalie BROOKS POWELL, Bolingbrook, IL (US); Santosh RAJASHEKAR, Bangalore (IN); Selvi JOHN, Bangalore (IN)

(21) Appl. No.: 19/206,591

(22) Filed: May 13, 2025

(51) Int. Cl. G06T 13/40; G06T 19/00; G16H 10/60

(52) U.S. Cl. CPC G06T 13/40; G06T 19/006; G16H 10/60; G06T 2210/41

(57) ABSTRACT

In some implementations, a computer system may receive historical patient data that includes one or more of a wearable device feed, a video recording of a rehabilitation session, or a medical report associated with a patient, and may anonymize the historical patient data to generate historical anonymized patient data. The computer system may create a personalized three-dimensional avatar based on the historical anonymized patient data, and may process the historical anonymized patient data, with a machine learning model, to predict one or more movements for rehabilitation of the patient. The computer system may update the personalized three-dimensional avatar based on the one or more movements to generate an animated avatar performing the one or more movements, and may provide the animated avatar to the patient in real-time through an augmented reality device.

### BACKGROUND

The field of physical rehabilitation generally involves helping patients through a variety of exercises and therapies. Rehabilitation instruction has traditionally been provided in various forms, and technology continues to evolve and expand the options available for aiding in the rehabilitation process.

### SUMMARY

Some aspects described herein relate to a method. The method may include receiving historical patient data that includes one or more of a wearable device feed, a video recording of a rehabilitation session, or a medical report associated with a patient, and anonymizing the historical patient data to generate historical anonymized patient data. The method may include creating a personalized three-dimensional avatar based on the historical anonymized patient data, and processing the historical anonymized patient data, with a machine learning model, to predict one or more movements for rehabilitation of the patient. The method may include updating the personalized three-dimensional avatar based on the one or more movements to generate an animated avatar performing the one or more movements, and providing the animated avatar to a medical professional for review. The method may include providing the animated avatar to the patient in real-time through an augmented reality device.

Some aspects described herein relate to a computer system. The computer system may include a processor set, one or more computer-readable storage media, and program instructions stored on the one or more computer-readable storage media to cause the processor set to perform operations. The operations may include receiving historical patient data that includes one or more of a wearable device feed, a video recording of a rehabilitation session, or a medical report associated with a patient, and anonymizing the historical patient data to generate historical anonymized patient data. The operations may include generating a knowledge corpus based on the historical anonymized patient data, and creating a personalized three-dimensional avatar based on the historical anonymized patient data. The operations may include processing the historical anonymized patient data, with a machine learning model, to predict one or more movements for rehabilitation of the patient, and updating the personalized three-dimensional avatar based on the one or more movements to generate an animated avatar performing the one or more movements. The operations may include providing the animated avatar to the patient in real-time through an augmented reality device.

Some aspects described herein relate to a computer program product. The computer program product may include one or more computer-readable storage media and program instructions stored on the one or more computer-readable storage media to perform operations. The operations may include receiving historical patient data that includes one or more of a wearable device feed, a video recording of a rehabilitation session, or a medical report associated with a patient, and anonymizing the historical patient data to generate historical anonymized patient data. The operations may include creating a personalized three-dimensional avatar based on the historical anonymized patient data, and processing the historical anonymized patient data, with a machine learning model, to predict one or more movements for rehabilitation of the patient. The operations may include updating the personalized three-dimensional avatar based on the one or more movements to generate an animated avatar performing the one or more movements, and providing the animated avatar to the patient in real-time through an augmented reality device. The operations may include receiving feedback from the patient during performance of the rehabilitation provided by the animated avatar, and adjusting the one or more movements of the animated avatar based on the feedback.

### BRIEF DESCRIPTION OF THE DRAWINGS

FIG. 1 is a diagram of an example computing environment for generating personalized three-dimensional avatars for patient rehabilitation, as described herein.

FIGS. 2A-2F are diagrams of an example associated with generating personalized three-dimensional avatars for patient rehabilitation.

FIG. 3 is a diagram illustrating an example of training and using a machine learning model.

FIG. 4 is a flowchart of an example process associated with generating personalized three-dimensional avatars for patient rehabilitation.

### DETAILED DESCRIPTION

The following detailed description of example implementations refers to the accompanying drawings. The same reference numbers in different drawings may identify the same or similar elements.

Rehabilitation after injury or surgery is a crucial process that requires patients to follow a progressive and personalized plan to recover safely and effectively. Traditional methods of rehabilitation, such as verbal guidance and static diagrams, may not provide patients with the clarity and customization needed for optimal recovery. Patients with various conditions, such as scoliosis, arthritis, or post-surgery recovery, often struggle to adjust their mobility patterns, which can lead to further injuries or complications. Current methods for rehabilitation often rely on manual observations and subjective assessments by medical professionals, which can be time-consuming and prone to errors. Moreover, patients may not receive immediate feedback and corrections, which can hinder their progress and increase the risk of re-injury.

Patients may not have access to real-time guidance and support outside of clinical settings, which can make it challenging to maintain a consistent and effective rehabilitation routine. The lack of personalized and adaptive rehabilitation plans can lead to a slower recovery process, reduced mobility, and decreased quality of life. Furthermore, the current methods for rehabilitation often fail to account for the varied and dynamic environments that patients encounter, which can impact their rehabilitation progress. For example, different surfaces, obstacles, and spatial layouts may require patients to adjust their mobility patterns. Thus, traditional methods for rehabilitating patients consume computing resources (e.g., processing resources, memory resources, communication resources, and/or the like), networking resources, and/or the like associated with failing to properly rehabilitate patients in a reasonable time, handling further injuries caused by patients improperly performing rehabilitation movements, generating subjective assessments of patients that are prone to errors, correcting the errors caused by the subjective assessments of patients, and/or the like.

Some implementations described herein provide a computer system for generating personalized three-dimensional avatars for patient rehabilitation. For example, the computer system may receive historical patient data that includes one or more of a wearable device feed, a video recording of a rehabilitation session, or a medical report associated with a patient, and may anonymize the historical patient data to generate historical anonymized patient data. The computer system may create a personalized three-dimensional avatar based on the historical anonymized patient data, and may process the historical anonymized patient data, with a machine learning model, to predict one or more movements for rehabilitation of the patient. The computer system may update the personalized three-dimensional avatar based on the one or more movements to generate an animated avatar performing the one or more movements, and may provide the animated avatar to the patient in real-time through an augmented reality device.

In this way, the computer system generates personalized three-dimensional avatars for patient rehabilitation. For example, the computer system may optimize rehabilitation guidance through real-time data analysis and predictive modeling, and may enhance the accuracy and efficiency of rehabilitation treatment. The computer system may utilize machine learning models and generative adversarial network models to analyze and generate personalized rehabilitation plans. The computer system may create personalized three-dimensional avatars for patients. The computer system may predict movements for rehabilitation and may update the avatars to perform the predicted movements. The computer system may provide the updated avatars to medical professionals for review, may receive modifications, and may modify the avatars based on the modifications to generate animated avatars. The computer system may provide the animated avatars to the patients in real-time through augmented reality devices, may receive feedback from the patients during performance of the rehabilitation, and may adjust the movements of the animated avatars based on the feedback. Thus, the computer system conserves computing resources, networking resources, and/or the like that would otherwise have been consumed by failing to properly rehabilitate patients in a reasonable time, handling further injuries caused by patients improperly performing rehabilitation movements, generating subjective assessments of patients that are prone to errors, correcting the errors caused by the subjective assessments of patients, and/or the like.

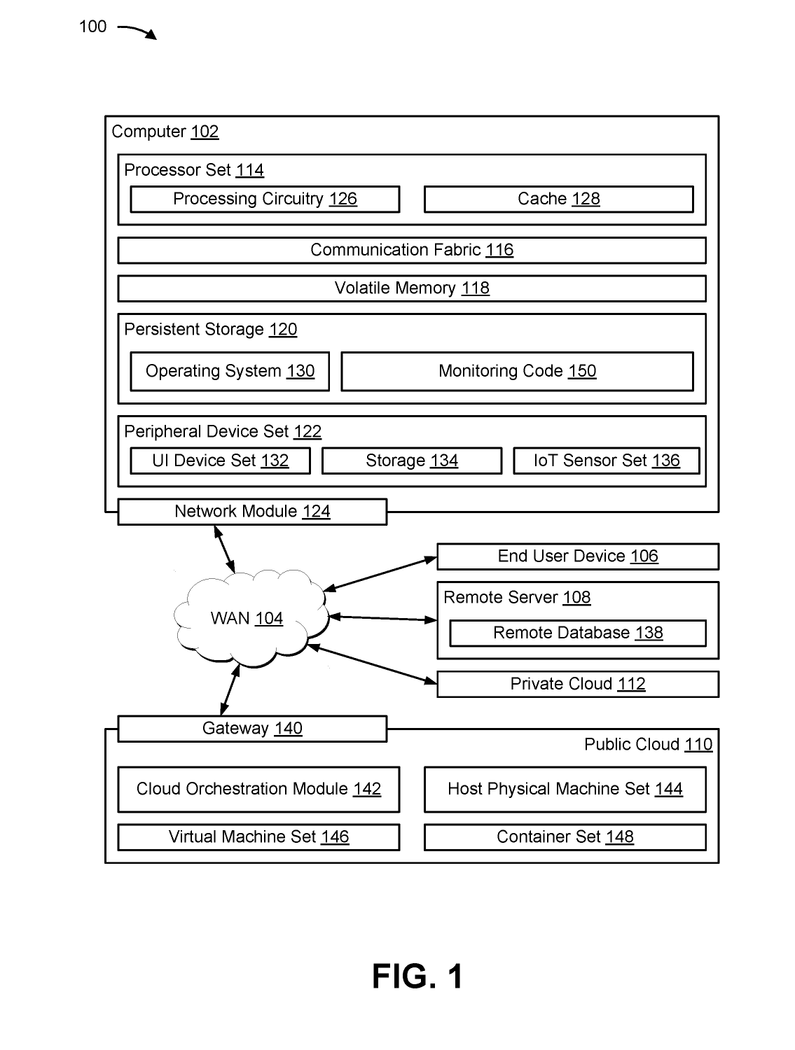

FIG. 1 is a diagram of an example computing environment 100 for generating personalized three-dimensional avatars for patient rehabilitation, as described herein.

Computing environment 100 contains an example of an environment for the execution of at least some of the computer code involved in performing the inventive methods, such as monitoring code 150. In addition to monitoring code 150, computing environment 100 includes, for example, a computer 102, a wide area network (WAN) 104, an end user device (EUD) 106, a remote server 108, a public cloud 110, and a private cloud 112. In this embodiment, computer 102 includes a processor set 114 (including processing circuitry 126 and a cache 128), a communication fabric 116, a volatile memory 118, persistent storage 120 (including an operating system 130 and monitoring code 150, as identified above), a peripheral device set 122 (including a user interface (UI) device set 132, storage 134, and an Internet of Things (IoT) sensor set 136), and a network module 124. Remote server 108 includes a remote database 138. Public cloud 110 includes a gateway 140, a cloud orchestration module 142, a host physical machine set 144, a virtual machine set 146, and a container set 148.

Computer 102 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network, or querying a database, such as remote database 138. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 102, to keep the presentation as simple as possible. Computer 102 may be located in a cloud, even though it is not shown in a cloud in FIG. 1. On the other hand, computer 102 is not required to be in a cloud except to any extent as may be affirmatively indicated.

Processor set 114 includes one, or more, computer processors of any type now known or to be developed in the future. Processing circuitry 126 may be distributed over multiple packages (for example, multiple, coordinated integrated circuit chips). Processing circuitry 126 may implement multiple processor threads and/or multiple processor cores. Cache 128 is memory that is located in the processor chip package(s) and is typically used for data or code that should be available for rapid access by the threads or cores running on processor set 114. Cache memories are typically organized into multiple levels depending upon relative proximity to the processing circuitry. Alternatively, some, or all, of the cache for the processor set may be located “off chip.” In some computing environments, processor set 114 may be designed for working with qubits and performing quantum computing.

Computer-readable program instructions are typically loaded onto computer 102 to cause a series of operational steps to be performed by processor set 114 of computer 102 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 128 and the other storage media discussed below. The program instructions, and associated data, are accessed by processor set 114 to control and direct performance of the inventive methods. In computing environment 100, at least some of the instructions for performing the inventive methods may be stored in monitoring code 150 in persistent storage 120.

Communication fabric 116 is the signal conduction path that allows the various components of computer 102 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

Volatile memory 118 is any type of volatile memory now known or to be developed in the future. Examples include dynamic type random access memory (RAM) or static type RAM. Typically, volatile memory 118 is characterized by random access, but this is not required unless affirmatively indicated. In computer 102, the volatile memory 118 is located in a single package and is internal to computer 102, but, alternatively or additionally, the volatile memory may be distributed over multiple packages and/or located externally with respect to computer 102.

Persistent storage 120 is any form of non-volatile storage for computers that is now known or to be developed in the future. The non-volatility of this storage means that the stored data is maintained regardless of whether power is being supplied to computer 102 and/or directly to persistent storage 120. Persistent storage 120 may be a read only memory (ROM), but typically at least a portion of the persistent storage allows writing of data, deletion of data, and re-writing of data. Some familiar forms of persistent storage include magnetic disks and solid-state storage devices. Operating system 130 may take any of several forms, such as various known proprietary operating systems or open source Portable Operating System Interface-type operating systems that employ a kernel.

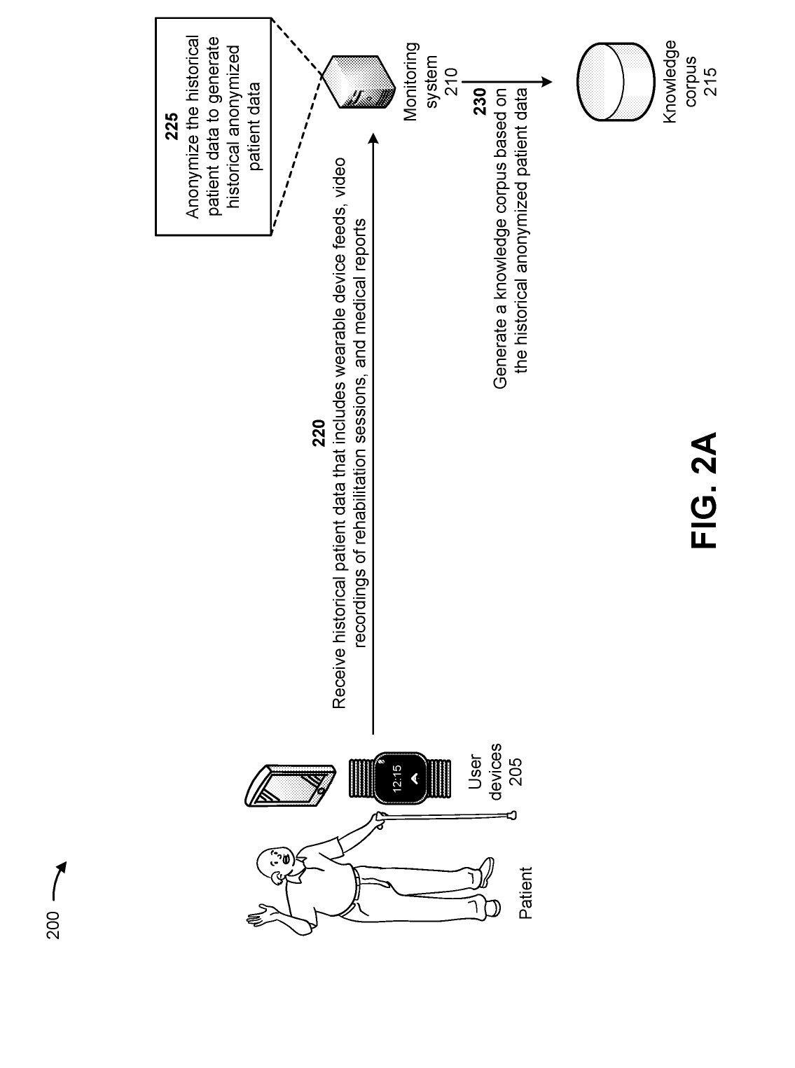

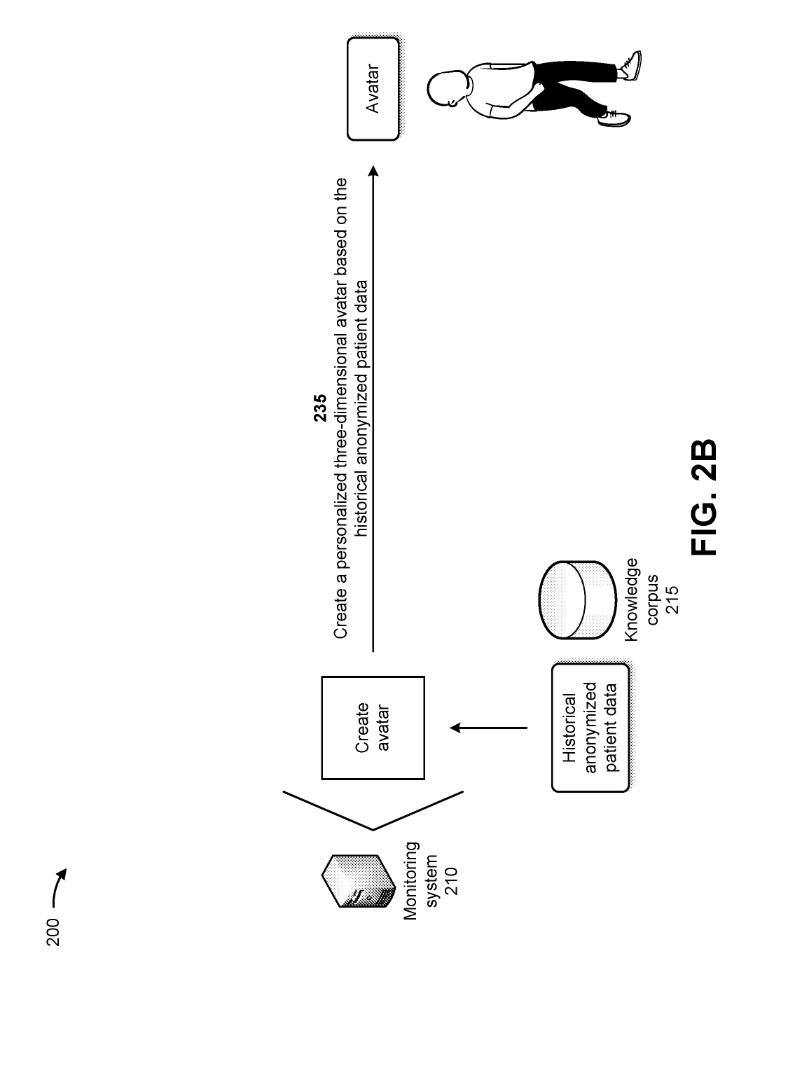

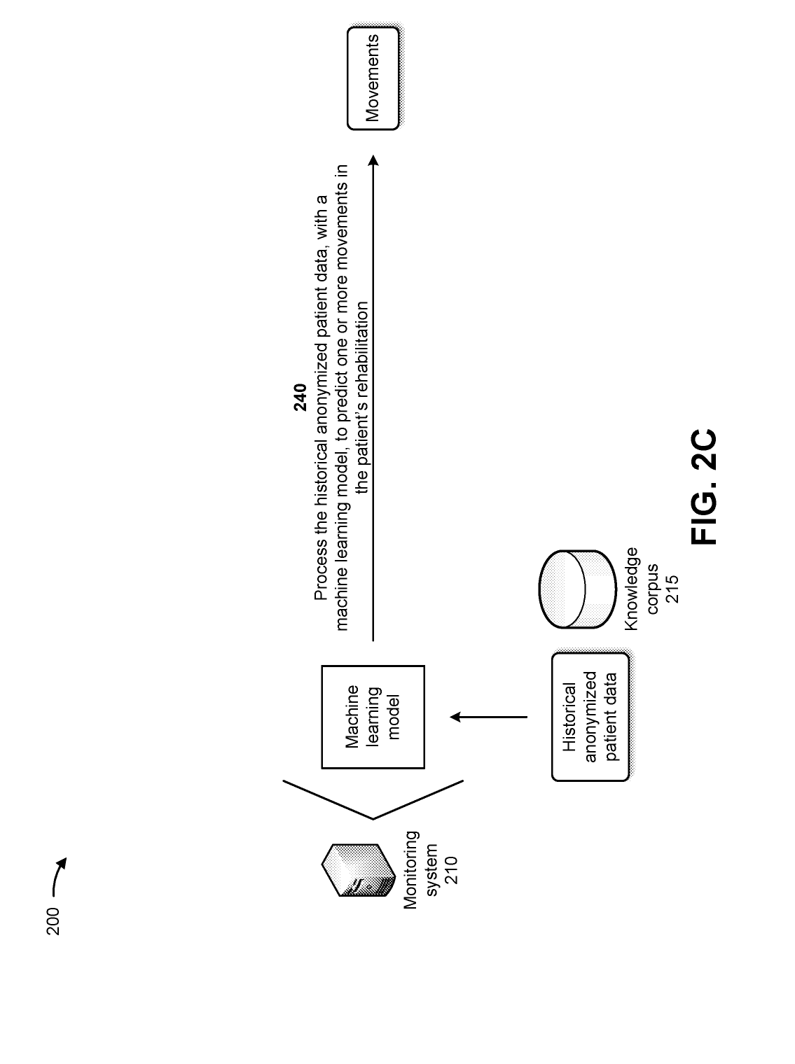

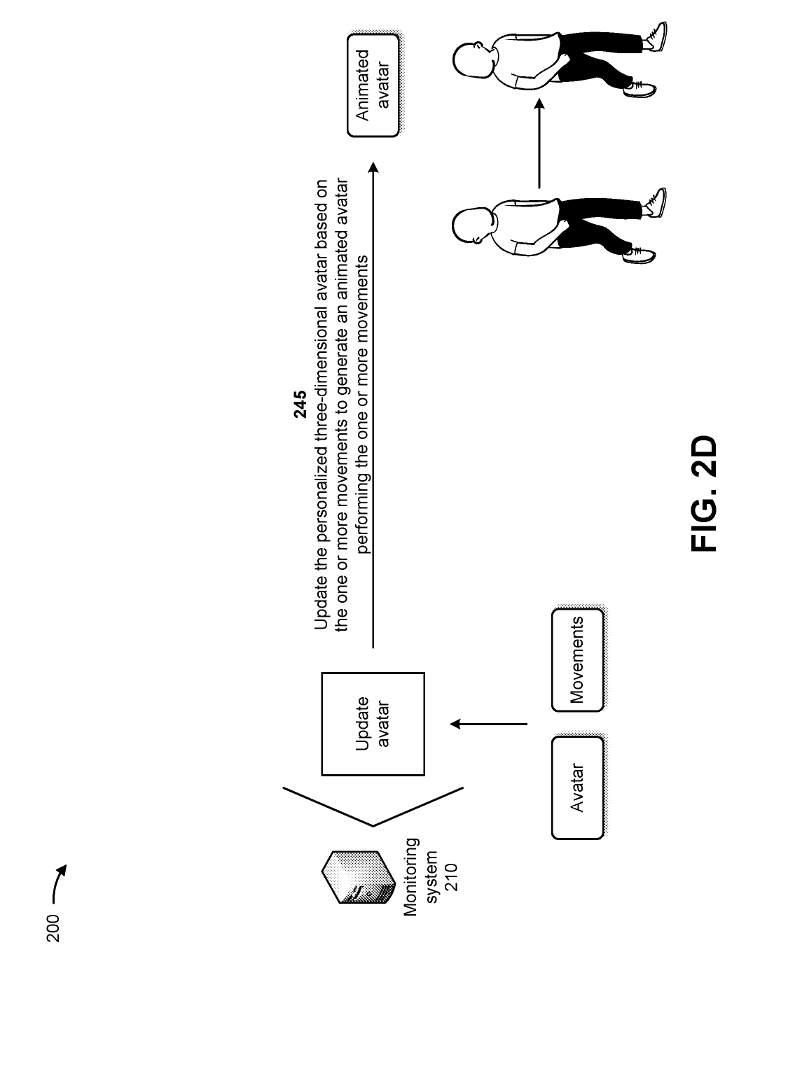

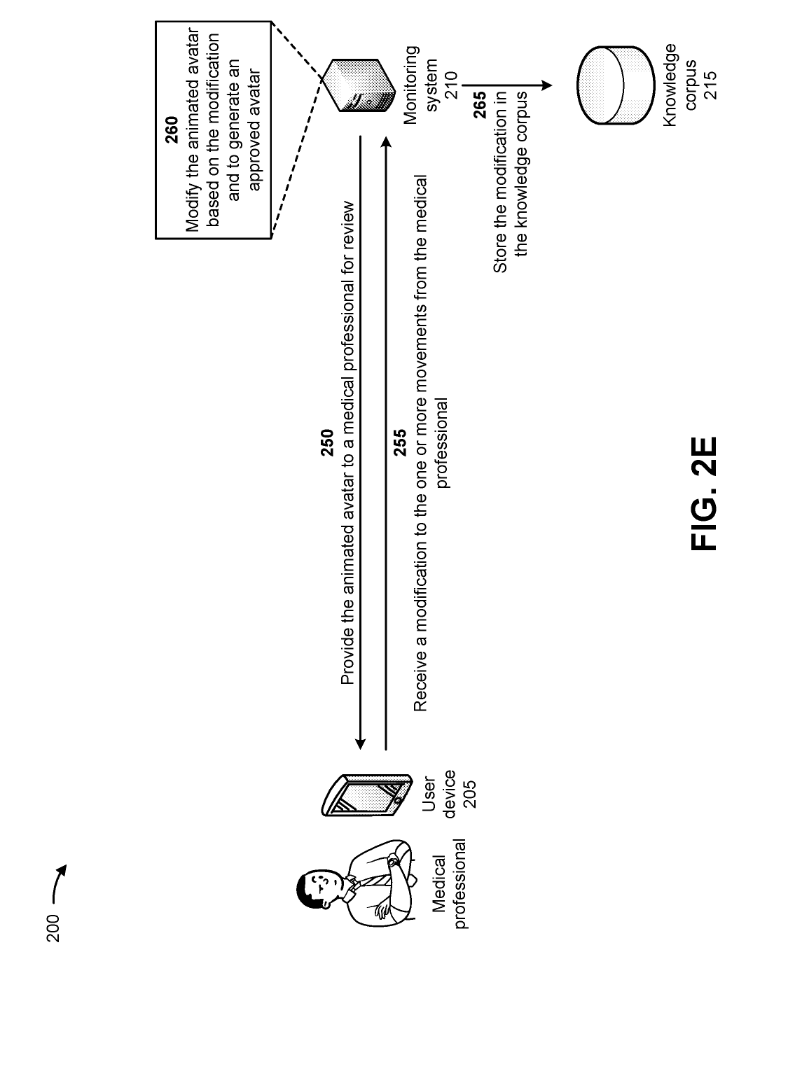

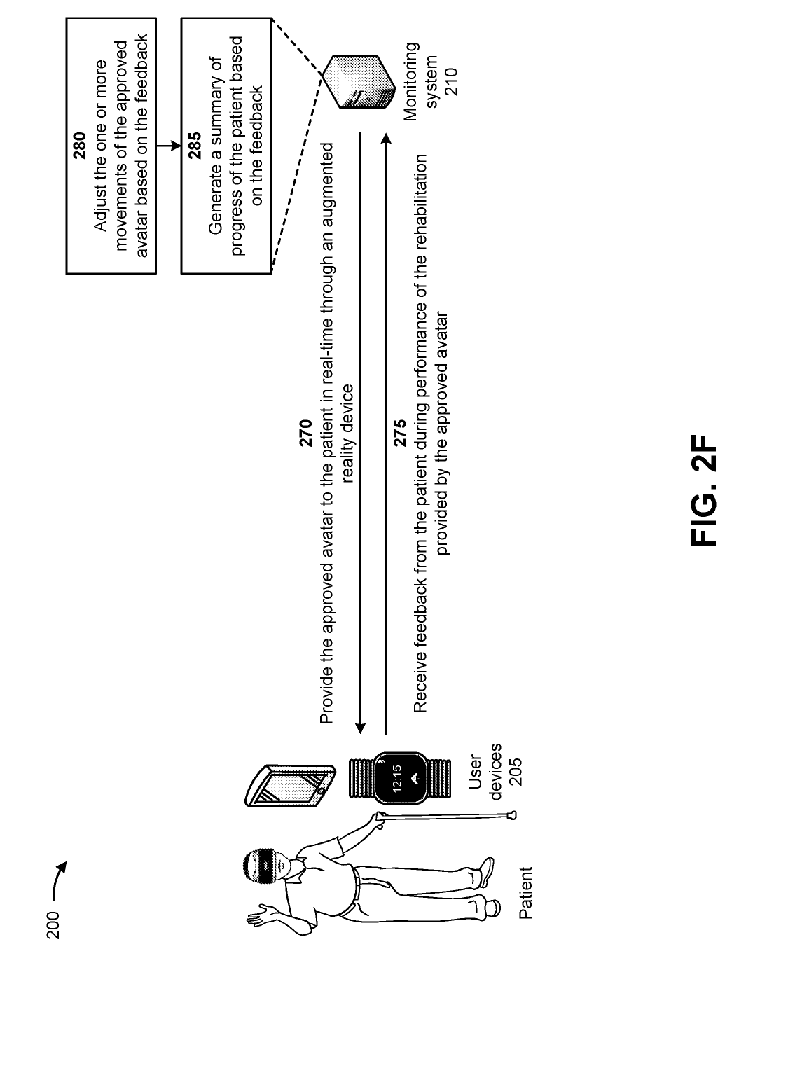

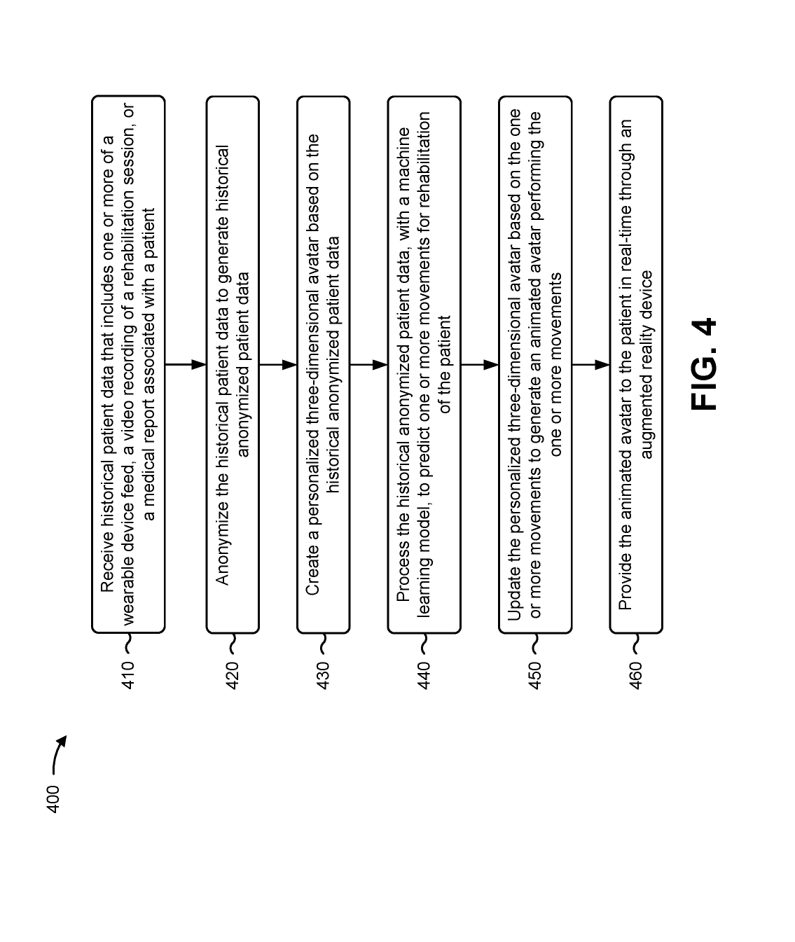

The code included in the monitoring code 150 typically includes at least some of the computer code involved in performing one or more operations described herein, such as the operations of implementation 200 in FIGS. 2A-2F and the process described in FIG. 4.

Peripheral device set 122 includes the set of peripheral devices of computer 102. Data communication connections between the peripheral devices and the other components of computer 102 may be implemented in various ways, such as Bluetooth® connections, Near-Field Communication (NFC) connections, connections made by cables (such as universal serial bus (USB) type cables), insertion-type connections (for example, secure digital (SD) card), connections made through local area communication networks and/or connections made through wide area networks such as the internet. In various embodiments, UI device set 132 may include components such as a display screen, speaker, microphone, wearable devices (such as goggles and smart watches), keyboard, mouse, printer, touchpad, game controllers, and/or haptic devices. Storage 134 is external storage, such as an external hard drive, or insertable storage, such as an SD card. Storage 134 may be persistent and/or volatile. In some embodiments, storage 134 may take the form of a quantum computing storage device for storing data in the form of qubits. In embodiments where computer 102 is required to have a large amount of storage (for example, where computer 102 locally stores and manages a large database), this storage may be provided by peripheral storage devices designed for storing very large amounts of data, such as a storage area network (SAN) that is shared by multiple, geographically distributed computers. IoT sensor set 136 is made up of sensors that can be used in Internet of Things applications. For example, one sensor may be a thermometer and another sensor may be a motion detector.

Network module 124 is the collection of computer software, hardware, and firmware that allows computer 102 to communicate with other computers through WAN 104. Network module 124 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 124 are performed on the same physical hardware device. In other embodiments (for example, embodiments that utilize software-defined networking (SDN)), the control functions and the forwarding functions of network module 124 are performed on physically separate devices, such that the control functions manage several different network hardware devices. Computer-readable program instructions for performing the inventive methods can typically be downloaded to computer 102 from an external computer or external storage device through a network adapter card or network interface included in network module 124.

WAN 104 is any wide area network (for example, the internet) capable of communicating computer data over non-local distances by any technology for communicating computer data, now known or to be developed in the future. In some embodiments, the WAN 104 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and/or edge servers.

EUD 106 is any computer system that is used and controlled by an end user (for example, a customer of an enterprise that operates computer 102), and may take any of the forms discussed above in connection with computer 102. EUD 106 typically receives helpful and useful data from the operations of computer 102. For example, in a hypothetical case where computer 102 is designed to provide a recommendation to an end user, this recommendation would typically be communicated from network module 124 of computer 102 through WAN 104 to EUD 106. In this way, EUD 106 can display, or otherwise present, the recommendation to an end user. In some embodiments, EUD 106 may be a client device, such as thin client, heavy client, mainframe computer, desktop computer and so on.

Remote server 108 is any computer system that serves at least some data and/or functionality to computer 102. Remote server 108 may be controlled and used by the same entity that operates computer 102. Remote server 108 represents the machine(s) that collect and store helpful and useful data for use by other computers, such as computer 102. For example, in a hypothetical case where computer 102 is designed and programmed to provide a recommendation based on historical data, this historical data may be provided to computer 102 from remote database 138 of remote server 108.

Public cloud 110 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 110 is performed by the computer hardware and/or software of cloud orchestration module 142. The computing resources provided by public cloud 110 are typically implemented by virtual computing environments that run on various computers making up the computers of host physical machine set 144, which is the universe of physical computers in and/or available to public cloud 110. The virtual computing environments (VCEs) typically take the form of virtual machines from virtual machine set 146 and/or containers from container set 148. These VCEs may be stored as images and may be transferred among and between the various physical machine hosts, either as images or after instantiation of the VCE. Cloud orchestration module 142 manages the transfer and storage of images, deploys new instantiations of VCEs, and manages active instantiations of VCE deployments. Gateway 140 is the collection of computer software, hardware, and firmware that allows public cloud 110 to communicate through WAN 104.

Some further explanation of VCEs will now be provided. VCEs can be stored as “images.” A new active instance of a VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 112 is similar to public cloud 110, except that the computing resources are only available for use by a single enterprise. While private cloud 112 is depicted as being in communication with WAN 104, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this example, public cloud 110 and private cloud 112 are both part of a larger hybrid cloud.

Cloud computing services and/or microservices (not separately shown in FIG. 1): private and public clouds 110 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider’s systems, and back. In some embodiments, cloud services may be configured and orchestrated according to an “as a service” technology paradigm where content is being presented to an internal or external customer in the form of a cloud computing service. As-a-service offerings typically provide endpoints with which various customers interface. These endpoints are typically based on a set of application programming interfaces (APIs). One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with such tasks. Another category is Software-as-a-Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

The number and arrangement of devices and networks shown in FIG. 1 are provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g., one or more devices) of the computing environment 100 may perform one or more functions described as being performed by another set of devices of the computing environment 100.

FIGS. 2A-2F are diagrams of an example 200 associated with generating personalized three-dimensional avatars for patient rehabilitation. As shown in FIGS. 2A-2F, the example 200 includes user devices 205 associated with a patient, a monitoring system 210, and a knowledge corpus 215. Each of the user devices 205 may correspond to the computer 102 and/or the end user device 106. The monitoring system 210 may correspond to the computer 102, the end user device 106, the remote server 108, a system provided by the public cloud 110, and/or a system provided by the private cloud 112. The knowledge corpus may correspond to the persistent storage 120, the remote database 138, a database, a table, and/or a list. Further details of the user devices 205, the monitoring system 210, and the knowledge corpus are provided elsewhere herein.

As shown in FIG. 2A, and by reference number 220, the monitoring system 210 may receive historical patient data that includes wearable device feeds, video recordings of rehabilitation sessions, and medical reports. For example, the monitoring system 210 may receive historical patient data through various methods, including manual entry, automated data transfer, and data integration with other systems. In some implementations, the monitoring system 210 may connect to various wearable devices (e.g., user devices 205 associated with patient), such as fitness trackers, smartwatches, or other health monitoring devices, using protocols like Bluetooth, Wi-Fi, or cellular networks. The monitoring system 210 may integrate APIs of the wearable devices to retrieve historical patient data, such as activity levels, heart rate, or other physiological parameters. Alternatively, or additionally, the patient or a healthcare provider may manually upload wearable device data to the monitoring system 210 through a web portal, a mobile application, or another interface. Alternatively, or additionally, the wearable devices may stream data in real-time to the monitoring system 210, allowing for continuous monitoring and updates.

In some implementations, cameras may be integrated with the monitoring system 210 to record and upload video footage of rehabilitation sessions with the patient. The patient or a healthcare provider may manually upload video files of rehabilitation sessions to the monitoring system 210 through a web portal, a mobile application, or another interface. Alternatively, or additionally, the video files may be stored in cloud storage services and integrated with the monitoring system 210 through APIs or other data exchange protocols. In some implementations, the monitoring system 210 may integrate with an electronic health records (EHRs) system to retrieve medical reports, such as clinical notes, diagnosis, and treatment plans associated with the patient. Alternatively, or additionally, a healthcare provider may manually enter the medical reports into the monitoring system 210 through a web portal, mobile application, or another interface. The patient or the healthcare provider may upload the medical reports as files to the monitoring system 210 through a web portal, mobile application, or another interface.

In some implementations, the historical patient data may include health parameters and physiological parameters associated with the patient. The health parameters may include vital signs of the patient (e.g., a blood pressure, a pulse rate, a body temperature, a respiratory rate, and oxygen saturation), a medical history of the patient (e.g., existing medical conditions, such as diabetes, hypertension, or cardiovascular disease), pain levels of the patient (e.g., assessments of pain intensity, location, and duration), sleep patterns of the patient (e.g., quality and duration of sleep, including sleep disorders or disturbances), a nutritional status of the patient (e.g., dietary habits, caloric intake, and hydration levels), mental health of the patient (e.g., assessments of anxiety, depression, or other mental health conditions), medication regimen of the patient (e.g., a list of prescribed medications, dosages, and potential side effects), and/or the like.

The physiological parameters may include a range of motion of the patient (e.g., measurements of joint mobility and flexibility), a muscle strength of the patient (e.g., assessments of muscle power and endurance), balance and coordination of the patient (e.g., evaluations of equilibrium, proprioception, and overall balance), gait patterns of the patient (e.g., analysis of walking patterns, including stride length, cadence, and velocity), neuromuscular function of the patient (e.g., assessments of nerve conduction, reflexes, and muscle tone), cardiorespiratory function of the patient (e.g., measures of heart rate, blood pressure, and oxygen consumption during exercise or physical activity), sensory function of the patient (e.g., evaluations of vision, hearing, and other sensory modalities), and/or the like.

In some implementations, the monitoring system 210 may standardize and integrate the historical patient data using data normalization (e.g., transforming data into a standardized format for easier analysis and processing), data aggregation (e.g., combining the data into a single, comprehensive dataset), data cleansing (e.g., identifying and correcting errors, inconsistencies, or incomplete data), data warehousing (e.g., storing integrated data in a centralized repository, such as the knowledge corpus 215, for analysis and reporting), and/or the like.

In some implementations, the monitoring system 210 may utilize a sensor fusion model that integrates data from multiple sensors placed on the patient’s body, such as accelerometers, gyroscopes, and pressure sensors. The sensor fusion model may provide data synchronization, noise filtering, complementary analysis, and weighted averaging to produce a unified and accurate model of the patient’s gait. By combining data from various sensors, the monitoring system 210 may detect subtle movement deviations and may provide real-time corrective feedback through an avatar. This comprehensive real-time gait analysis may enhance the effectiveness of the rehabilitation program by ensuring precise and adaptive guidance.

As further shown in FIG. 2A, and by reference number 225, the monitoring system 210 may anonymize the historical patient data to generate historical anonymized patient data. For example, the monitoring system 210 may anonymize the historical patient data to generate the historical anonymized patient data by employing various techniques to remove or obscure personally identifiable information (PII) associated with the patient. Anonymization is a process that transforms personal data into data that cannot be linked to an individual patient, without significantly affecting the usefulness of the data for analysis and other legitimate purposes. In some implementations, the monitoring system 210 may apply data masking techniques to conceal identifying information from the historical patient data, such as a patient name, an address, a telephone number, and a social security number. For example, the monitoring system 210 may replace the patient’s name with a pseudonym or a random string of characters.

Alternatively, or additionally, the monitoring system 210 may utilize tokenization to replace sensitive information in the historical patient data with nonsensitive, unique identifiers, such as tokens or codes. For example, the monitoring system 210 may replace the patient’s identification number with a unique token that cannot be linked to the patient. Alternatively, or additionally, the monitoring system 210 may encrypt the historical patient data to protect the historical patient data from unauthorized access. The encryption may transform the historical patient data into an unreadable format, making the data unintelligible to unauthorized parties. Alternatively, or additionally, the monitoring system 210 may apply hashing algorithms to transform sensitive information of the historical patient data, such as patient identifiers or birthdates, into fixed-length strings of characters that cannot be reversed or decrypted. Alternatively, or additionally, the monitoring system 210 may de-identify the historical patient data by removing direct and indirect identifiers, such as a patient name, address, telephone number, and social security number. The monitoring system 210 may also remove any data that can be used to infer the patient’s identity, such as unique device identifiers or Internet protocol (IP) addresses.

Alternatively, or additionally, the monitoring system 210 may aggregate the historical patient data to prevent individual patients from being identified. For example, the monitoring system 210 may group data by age range or geographic region to conceal individual identities. Alternatively, or additionally, the monitoring system 210 may perturb the historical patient data by adding noise or randomly altering certain values to prevent individual patients from being identified. Perturbation can be applied to variables like age, date of birth, or zip code. Alternatively, or additionally, the monitoring system 210 may suppress certain data elements that are deemed too sensitive or identifying. For example, the monitoring system 210 may remove any data related to the patient’s mental health or genetically sensitive information. Alternatively, or additionally, the monitoring system 210 may use pseudonymization to replace identifying information with pseudonyms, such as artificial patient identifiers or fictional names. Alternatively, or additionally, the monitoring system 210 may employ secure key management practices to ensure that any cryptographic keys used to encrypt or decrypt the historical patient data are properly stored and managed to prevent unauthorized access.

As further shown in FIG. 2A, and by reference number 230, the monitoring system 210 may generate the knowledge corpus 215 based on the historical anonymized patient data. For example, the monitoring system 210 may generate the knowledge corpus 215 based on the historical anonymized patient data by employing various techniques to analyze, process, and transform the data into a structured and meaningful repository of knowledge. In some implementations, the monitoring system 210 may standardize and integrate the historical anonymized patient data using data preprocessing (e.g., preprocessing the historical anonymized patient data to ensure it is accurate, complete, and consistent), data normalization (e.g., converting data types, scaling numerical values, and encoding categorical variables), data transformation (e.g., utilizing dimensionality reduction, feature extraction, and time-series analysis to extract relevant features and patterns from the historical anonymized patient data), and/or the like. Alternatively, or additionally, the monitoring system 210 may utilize pattern detection to detect patterns and relationships within the historical anonymized patient data. This may include utilizing machine learning models (e.g., such as decision trees, clustering, or regression analysis, to identify patterns and relationships), statistical analysis (e.g., applying hypothesis testing, correlation analysis, or confidence intervals to identify significant patterns and relationships), data mining (e.g., utilizing association rule mining, frequent pattern mining, or sequence mining to discover hidden patterns and relationships), and/or the like.

Alternatively, or additionally, the monitoring system 210 may utilize knowledge representation to represent the detected patterns and relationships in a structured and meaningful way, using ontologies (e.g., creating a formal representation of a knowledge domain, using concepts, relationships, and axioms), knowledge graphs (e.g., representing knowledge as a network of interconnected entities, relationships, and attributes), frames (e.g., to represent knowledge as a set of attributes and their values), and/or the like. In some implementations, the monitoring system 210 may integrate the represented knowledge into a cohesive and comprehensive knowledge corpus 215 using merging (e.g., combining multiple sources of knowledge into a single, unified repository), alignment (e.g., aligning the knowledge corpus 215 with external sources of knowledge, such as standards, guidelines, or domain-specific knowledge), and/or the like. Alternatively, or additionally, the monitoring system 210 may validate the knowledge corpus 215 to ensure the knowledge corpus 215 is accurate, complete, and consistent using techniques such as evaluation metrics (e.g., precision, recall, or F1-score), expert review (e.g., having domain experts review and validate the knowledge corpus 215), and/or the like. Alternatively, or additionally, the monitoring system 210 may maintain and update the knowledge corpus 215 to ensure the knowledge corpus 215 remains relevant and effective. This may include utilizing techniques such as incremental learning (e.g., updating the knowledge corpus 215 incrementally, as new data becomes available), active learning (e.g., selecting the most informative or uncertain samples for manual annotation and update), and/or the like. By applying these techniques, the monitoring system 210 may generate a comprehensive and accurate knowledge corpus 215. The monitoring system 210 may utilize the knowledge corpus 215 to train machine learning models, support data-driven quality improvement initiatives, and enable healthcare organizations to make data-driven decisions.

As shown in FIG. 2B, and by reference number 235, the monitoring system 210 may create a personalized three-dimensional avatar based on the historical anonymized patient data. For example, the monitoring system 210 may create a personalized three-dimensional avatar based on the historical anonymized patient data by employing various techniques to analyze, process, and transform the data into a visual representation of the patient. The monitoring system 210 may analyze the historical anonymized patient data to identify relevant information that can be used to create the personalized three-dimensional avatar. This may include using data mining techniques, such as clustering or decision trees, to identify patterns and relationships in the data; using machine learning models, such as a convolutional neural network (CNN) model or a long short-term memory (LSTM) model, to analyze the data and make predictions about the patient’s physical characteristics; applying statistical methods, such as hypothesis testing or confidence intervals, to identify significant trends and patterns in the data; and/or the like.

In some implementations, the monitoring system 210 may estimate the patient’s body shape based on the analyzed data. For example, the monitoring system 210 may utilize parametric models, such as linear or nonlinear regression, to estimate the patient’s body shape based on their height, weight, and other characteristics, may utilize three-dimensional scanning data or depth images to estimate the patient’s body shape, may utilize generalized linear models, such as logistic or log-linear regression models, to estimate the patient’s body shape, and/or the like. The monitoring system 210 may estimate the patient’s skeletal joint positions and movements based on the analyzed data. For example, the monitoring system 210 may utilize kinematic analysis to estimate the patient’s joint positions and movements based on gait, posture, and other movement patterns; may utilize machine learning models, such as neural network or regression analysis models, to estimate the patient’s joint positions and movements based on their walking patterns, muscle activity, and other factors; may utilize computer vision techniques, such as motion capture or optical flow analysis, to estimate the patient’s joint positions and movements; and/or the like.

In some implementations, the monitoring system 210 may simulate soft tissue deformation based on the estimated body shape and skeletal joint positions. For example, the monitoring system 210 may utilize finite element analysis to simulate the deformation of soft tissues, such as skin, muscles, and fat, based on the patient’s body shape and joint positions; may utilize soft tissue modeling techniques, such as mesh-based or physics-based modeling, to simulate the deformation of soft tissues; may utilize machine learning models, such as neural network or regression analysis models, to learn relationships between soft tissue deformation and body shape, joint positions, and other factors; and/or the like.

The monitoring system 210 may create the personalized three-dimensional avatar based on the estimated body shape, skeletal joint positions, and soft tissue deformation. For example, the monitoring system 210 may utilize three-dimensional rendering techniques to create the personalized three-dimensional avatar based on the estimated body shape and joint positions; may apply textures and shading effects to the personalized three-dimensional avatar to create a realistic and personalized representation of the patient; may utilize animation techniques, such as keyframe animation or physics-based animation, to animate the personalized three-dimensional avatar and simulate the patient’s movements and activities; and/or the like. In some implementations, the monitoring system 210 may refine the personalized three-dimensional avatar based on additional data or feedback. For example, the monitoring system 210 may validate the accuracy and realism of the personalized three-dimensional avatar against real-world data, such as photographs or medical imaging data, may refine the personalized three-dimensional avatar based on feedback from medical professionals, patients, or caregivers, and/or the like.

As shown in FIG. 2C, and by reference number 240, the monitoring system 210 may process the historical anonymized patient data, with a machine learning model, to predict one or more movements in the patient’s rehabilitation. For example, the monitoring system 210 may prepare the historical anonymized patient data for processing by the machine learning model by utilizing data normalization, data feature extraction, data labeling, and/or the like. In some implementations, the monitoring system 210 may select a suitable machine learning model for predicting movements. For example, the monitoring system 210 may utilize a regression model (e.g., a linear regression model or a logistic regression model) to predict continuous or categorical movement variables, may utilize a classification model (e.g., a decision tree model or a random forest model) to predict categorical movement variables, may utilize a deep learning model (e.g., a neural network model or a CNN model) to predict complex movement patterns, and/or the like.

In some implementations, the monitoring system 210 may train the machine learning model using historical data associated with previous patients. For example, the monitoring system 210 may train the machine learning model using labeled data to learn the relationships between input features and movement predictions, may train the machine learning model using unlabeled data to discover patterns and relationships in the data, may train the machine learning model using trial and error to learn optimal movement predictions, and/or the like. The monitoring system 210 may evaluate the performance of the trained machine learning model using metrics, such as accuracy (e.g., measuring the proportion of correct movement predictions), precision (e.g., measuring the proportion of true positive movement predictions), and recall (e.g., measuring the proportion of true positive movement predictions among actual movements). In some implementations, the monitoring system 210 process the historical anonymized patient data, with the trained machine learning model, to predict the one or more movements in the patient’s rehabilitation. For example, the monitoring system 210 may predict future movements based on the historical anonymized patient data, and may predict past movements based on current patient data.

As shown in FIG. 2D, and by reference number 245, the monitoring system 210 may update the personalized three-dimensional avatar based on the one or more movements to generate an animated avatar performing the one or more movements. For example, the monitoring system 210 may update the personalized three-dimensional avatar based on the predicted movements to generate the animated avatar performing the predicted movements. In some implementations, the monitoring system 210 may update the skeleton of the personalized three-dimensional avatar to reflect the predicted movements. For example, the monitoring system 210 may update the positions of the avatar’s joints to reflect the predicted movements, may update the lengths of the avatar’s bones to reflect the predicted movements, and/or the like. In some implementations, the monitoring system 210 may deform the mesh of the personalized three-dimensional avatar to reflect the predicted movements. For example, the monitoring system 210 may deform the mesh using linear blend skinning techniques, may deform the mesh using physically-based techniques (e.g., finite element analysis), and/or the like.

In some implementations, the monitoring system 210 may animate the personalized three-dimensional avatar to reflect the predicted movements. For example, the monitoring system 210 may animate the avatar using keyframe techniques, may animate the avatar using physics-based techniques (e.g., rigid body dynamics), and/or the like. In some implementations, the monitoring system 210 may render the animated personalized three-dimensional avatar to create a realistic and engaging visual representation of the patient’s movements. For example, the monitoring system 210 may render the avatar in real-time to provide immediate feedback, may render the avatar offline to provide more detailed and realistic visuals.

As shown in FIG. 2E, and by reference number 250, the monitoring system 210 may provide the animated avatar to a medical professional for review. For example, the monitoring system 210 may provide the animated avatar to a medical professional (e.g., via a user device 205) for review through various channels. In some implementations, the monitoring system 210 may provide a web-based interface that allows the medical professional to access and review the animated avatar. The interface may include features such as avatar playback (e.g., allowing the medical professional to play back the animated avatar and review the movements), avatar manipulation (e.g., allowing the medical professional to manipulate the animated avatar, such as zooming, panning, or rotating the avatar), and annotation tools (e.g., allowing the medical professional to annotate the animated avatar with notes or comments). Alternatively, or additionally, the monitoring system 210 may provide a mobile application that allows the medical professional to access and review the animated avatar on a mobile user device 205. The mobile application may include features such as avatar playback, avatar manipulation, and annotation tools. Alternatively, or additionally, the monitoring system 210 may provide a virtual reality (VR) interface or an augmented reality (AR) interface that allows the medical professional to interact with the animated avatar in a more immersive environment. The virtual reality interface or the augmented reality interface may include features such as allowing the medical professional to interact with the animated avatar in a more immersive environment (e.g., walking around the avatar or manipulating the avatar’s movements).

As further shown in FIG. 2E, and by reference number 255, the monitoring system 210 may receive a modification to the one or more movements from the medical professional. For example, the monitoring system 210 may receive a modification to the one or more movements from the medical professional through various channels. In some implementations, the monitoring system 210 may receive the modification through a web-based interface, where the medical professional can input the modification using a keyboard, mouse, or other input devices. Alternatively, or additionally, the monitoring system 210 may receive the modification through a mobile application, where the medical professional can input the modification using a touch screen or other input devices. Alternatively, or additionally, the monitoring system 210 may receive modification through a virtual reality interface or an augmented reality interface, where the medical professional can input the modification using gestures, voice commands, or other input methods.

In some implementations, the modification may include a change to the movement pattern, such as a change to an amplitude or a frequency of the one or more movements. Alternatively, or additionally the modification may include a change to a duration of the one or more movements, such as increasing or decreasing a length of time the one or more movements are performed. Alternatively, or additionally the modification may include a change to an intensity of the one or more movements, such as increasing or decreasing a force or a velocity of the one or more movements.

As further shown in FIG. 2E, and by reference number 260, the monitoring system 210 may modify the animated avatar based on the modification and to generate an approved avatar. For example, the monitoring system 210 may modify the animated avatar based on the modification received from the medical professional. After the modification, the animated avatar may be considered an approved avatar. In some implementations, the monitoring system 210 may utilize animation blending to modify the animated avatar and to generate the approved avatar. For example, the monitoring system 210 may blend the animated avatar with the modified animation (e.g., based on the modification) to create a smooth transition between the animated avatar and the approved avatar. In some implementations, the monitoring system 210 may utilize animation warping to modify the animated avatar and to generate the approved avatar. For example, the monitoring system 210 may warp the animated avatar to match the modified movement patterns. In some implementations, the monitoring system 210 may utilize animation retargeting to modify the animated avatar and to generate the approved avatar. For example, the monitoring system 210 may retarget the animated avatar to match the modified movement patterns and intensity. The modified animated avatar (e.g., the approved avatar) may be generated based on the modified movement patterns, duration, and intensity, and may be utilized for future reference or shared with the patient or other medical professionals.

In some implementations, the monitoring system 210 may utilize a generative adversarial network (GAN) model to modify the animated avatar based on the modification and to generate the approved avatar. For example, the monitoring system 210 may employ a GAN architecture that includes two neural networks: a generator network and a discriminator network. The generator network may receive a random noise vector and the modified movement patterns as input and may generate a modified animated avatar. The discriminator network may receive the generated avatar and the original animated avatar as input and may evaluate the similarity between the two. The generator network may use a CNN or a recurrent neural network (RNN) architecture to generate the avatar. The discriminator network may use a CNN or an RNN architecture to evaluate the similarity. The GAN model may utilize a loss function to evaluate the performance of the generator network and the discriminator network. The loss function may be a combination of the generator loss and the discriminator loss.

The GAN model may be trained using a dataset of animated avatars and their corresponding movement patterns. The generator network may be trained to generate avatars that are indistinguishable from the original avatars, while the discriminator network may be trained to distinguish between the generated avatars and the original avatars. The generator network and the discriminator network may be trained together in an adversarial manner. Once the GAN model is trained, the monitoring system 210 may utilize the GAN model to modify the animated avatar based on the modification. The generator network may receive the modified movement patterns as input and may generate a modified animated avatar. The discriminator network may evaluate the modified avatar and the original avatar to determine the similarity between the two. If the discriminator network determines that the modified avatar is similar to the original avatar, the monitoring system 210 may generate the approved avatar. Utilizing the GAN model to modify the animated avatar may provide improved accuracy (e.g., the GAN model may generate avatars that are highly accurate and realistic), increased efficiency (e.g., the GAN model may generate avatars quickly and efficiently), an flexibility (e.g., the GAN model may generate avatars with a wide range of movement patterns and styles, allowing for greater flexibility and customization).

As further shown in FIG. 2E, and by reference number 265, the monitoring system 210 may store the modification in the knowledge corpus 215. For example, the monitoring system 210 may store the modification in the knowledge corpus 215 along with historical anonymized patient data. The modification may be stored in the knowledge corpus 215 as a new entry that includes a modification timestamp (e.g., a timestamp indicating when the modification was made), a modification type (e.g., a description of the type of modification made, such as a change to movement patterns or intensity), and modification values (e.g., specific values of the modification, such as a new amplitude or a new frequency of the movements).

As shown in FIG. 2F, and by reference number 270, the monitoring system 210 may provide the approved avatar to the patient in real-time through an augmented reality device. For example, the monitoring system 210 may provide the approved avatar to the patient in real-time through an augmented reality device (e.g., augmented reality glasses) or virtual reality device (e.g., virtual reality glasses). In some implementations, the monitoring system 210 may connect to an augmented reality device, such as a headset or tablet, through a secure network connection, and may transmit the approved avatar to the augmented reality device in real-time, using a data communication protocol such as Wi-Fi or Bluetooth. The augmented reality device may receive the approved avatar and may render the approved avatar in real-time, using the augmented reality device’s camera and display to superimpose the approved avatar onto the patient’s real-world environment. The patient may manipulate the approved avatar in real-time, using hand gestures or voice commands to adjust the approved avatar’s movements and positions.

Using an augmented reality device to provide the approved avatar to the patient in real-time may provide a highly engaging and interactive experience, and may encourage the patient to participate in the rehabilitation exercises and track progress. The augmented reality device may provide real-time feedback to the patient and the medical professional, and may allow for rapid adjustments to the approved avatar and improved rehabilitation outcomes. The augmented reality device may automate many aspects of the rehabilitation process, reducing the need for manual intervention and allowing medical professionals to focus on providing high-quality care.

In some implementations, the monitoring system 210 may evaluate the patient’s surrounding context, such as a surface on which the patient is walking or a spatial layout, and may adjusts the avatar’s movements accordingly. For example, if the patient is walking on a slope or uneven ground, the monitoring system 210 may modify the avatar’s movement patterns to ensure the recommended exercises are suitable and safe for the patient’s real-world conditions. This real-time environmental context adaptation helps promote safer rehabilitation by tailoring the exercises to the actual conditions encountered by the patient.

As further shown in FIG. 2F, and by reference number 275, the monitoring system 210 may receive feedback from the patient during performance of the rehabilitation provided by the approved avatar. For example, the monitoring system 210 may receive feedback from the patient (e.g., via the augmented reality device) during performance of the rehabilitation provided by the approved avatar. For example, the augmented reality device and wearable devices may collect sensor data from the patient’s movements, such as accelerometer and gyroscope data, to track the patient’s progress and movements. The patient may provide feedback to the monitoring system 210, either through voice commands or manual input to the augmented reality device or another user device 205, to indicate pain or discomfort during the rehabilitation exercises. In some implementations, the augmented reality device may transmit the patient’s feedback to the monitoring system 210 in real-time, using the same data communication protocol used to transmit the approved avatar from the monitoring system 210 to the augmented reality device.

As further shown in FIG. 2F, and by reference number 280, the monitoring system 210 may adjust the one or more movements of the approved avatar based on the feedback. For example, the monitoring system 210 analyze the patient’s movements and the feedback to identify areas where the patient is experiencing pain or discomfort. The monitoring system 210 may adjust the approved avatar’s movements to accommodate the patient’s needs based on the analysis. For example, the monitoring system 210 may modify the movement patterns or may reduce an intensity and a duration of the exercises. The monitoring system 210 may update the approved avatar in real-time to generate a revised avatar, and may transmit the revised avatar to the augmented reality device for rendering.

As further shown in FIG. 2F, and by reference number 285, the monitoring system 210 may generate a summary of progress of the patient based on the feedback. For example, the monitoring system 210 may analyze the patient’s movement data and feedback to identify trends and patterns in the patient’s progress. The monitoring system 210 may calculate the patient’s progress based on the analysis, either by measuring the patient’s improvement in range of motion or strength, or by tracking the patient’s ability to complete exercises without pain or discomfort. The monitoring system 210 may generate a summary of the patient’s progress, either in the form of a written report or a graphical representation. In some implementations, the monitoring system 210 may provide the summary of the patient’s progress to user devices 205 associated with the patient and/or the medical professional. The summary may provide the patient and/or the medical professional with a clear understanding of the patient’s rehabilitation progress.

In this way, the monitoring system 210 generates personalized three-dimensional avatars for patient rehabilitation. For example, the monitoring system 210 may optimize rehabilitation guidance through real-time data analysis and predictive modeling, and may enhance the accuracy and efficiency of rehabilitation treatment. The monitoring system 210 may utilize machine learning models and generative adversarial network models to analyze and generate personalized rehabilitation plans. The monitoring system 210 may create personalized three-dimensional avatars for patients. The monitoring system 210 may predict movements for rehabilitation and may update the avatars to perform the predicted movements. The monitoring system 210 may provide the updated avatars to medical professionals for review, may receive modifications, and may modify the avatars based on the modifications to generate animated avatars. The monitoring system 210 may provide the animated avatars to the patients in real-time through augmented reality devices, may receive feedback from the patients during performance of the rehabilitation, and may adjust the movements of the animated avatars based on the feedback. Thus, the monitoring system 210 conserves computing resources, networking resources, and/or the like that would otherwise have been consumed by failing to properly rehabilitate patients in a reasonable time, handling further injuries caused by patients improperly performing rehabilitation movements, generating subjective assessments of patients that are prone to errors, correcting the errors caused by the subjective assessments of patients, and/or the like.

As indicated above, FIGS. 2A-2F are provided as an example. Other examples may differ from what is described with regard to FIGS. 2A-2F. The number and arrangement of devices shown in FIGS. 2A-2F are provided as an example. In practice, there may be additional devices, fewer devices, different devices, or differently arranged devices than those shown in FIGS. 2A-2F. Furthermore, two or more devices shown in FIGS. 2A-2F may be implemented within a single device, or a single device shown in FIGS. 2A-2F may be implemented as multiple, distributed devices. Additionally, or alternatively, a set of devices (e.g., one or more devices) shown in FIGS. 2A-2F may perform one or more functions described as being performed by another set of devices shown in FIGS. 2A-2F.

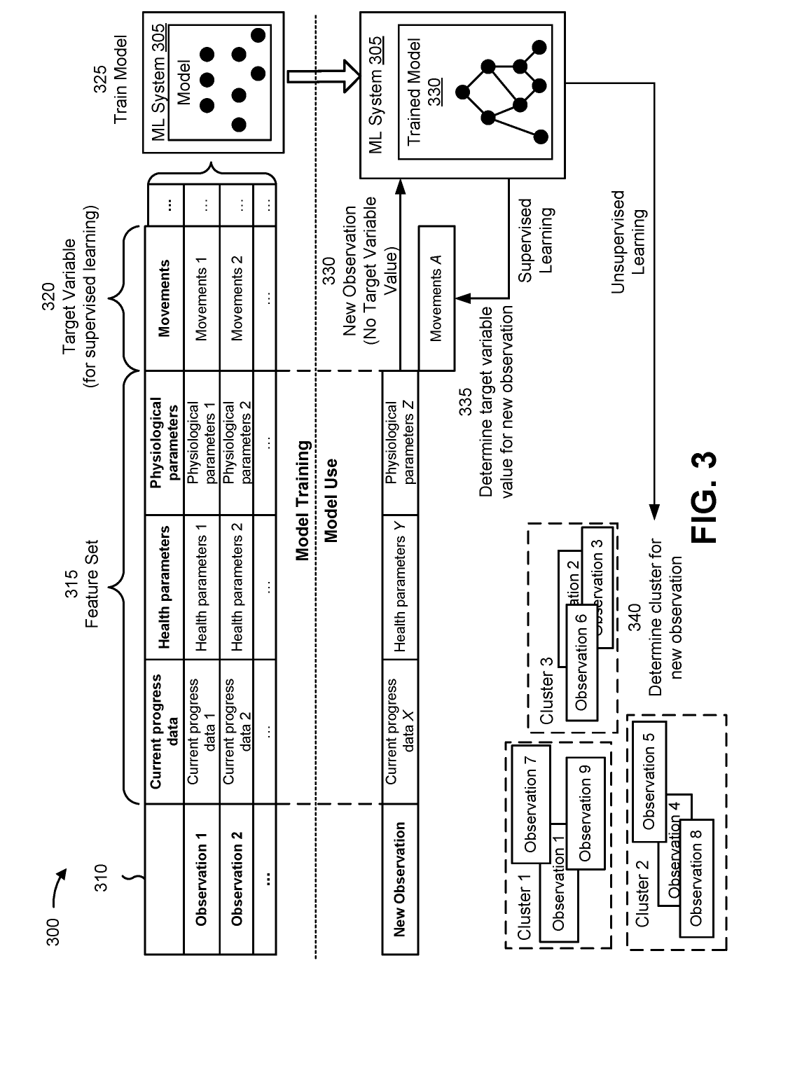

FIG. 3 is a diagram illustrating an example 300 of training and using a machine learning model. The machine learning model training and usage described herein may be performed using a machine learning system. The machine learning system may include or may be included in a computing device, a server, a cloud computing environment, and/or the like, such as the monitoring system 210 described in more detail elsewhere herein.

As shown by reference number 305, a machine learning model may be trained using a set of observations. The set of observations may be obtained from historical data, such as data gathered during one or more processes described herein. In some implementations, the machine learning system may receive the set of observations (e.g., as input) from the monitoring system 210, as described elsewhere herein.

As shown by reference number 310, the set of observations includes a feature set. The feature set may include a set of variables, and a variable may be referred to as a feature. A specific observation may include a set of variable values (or feature values) corresponding to the set of variables. In some implementations, the machine learning system may determine variables for a set of observations and/or variable values for a specific observation based on input received from the monitoring system 210. For example, the machine learning system may identify a feature set (e.g., one or more features and/or feature values) by extracting the feature set from structured data, by performing natural language processing to extract the feature set from unstructured data, by receiving input from an operator, and/or the like.

As an example, a feature set for a set of observations may include a first feature of current progress data, a second feature of health parameters, a third feature of physiological parameters, and so on. As shown, for a first observation, the first feature may have a value of current progress data 1, the second feature may have a value of health parameters 1, the third feature may have a value of physiological parameters 1, and so on. These features and feature values are provided as examples and may differ in other examples.

As shown by reference number 315, the set of observations may be associated with a target variable. The target variable may represent a variable having a numeric value, may represent a variable having a numeric value that falls within a range of values or has some discrete possible values, may represent a variable that is selectable from one of multiple options (e.g., one of multiple classes, classifications, labels, and/or the like), may represent a variable having a Boolean value, and/or the like. A target variable may be associated with a target variable value, and a target variable value may be specific to an observation. In example 300, the target variable may be entitled “movements” and may include a value of movements 1 for the first observation.

The target variable may represent a value that a machine learning model is being trained to predict, and the feature set may represent the variables that are input to a trained machine learning model to predict a value for the target variable. The set of observations may include target variable values so that the machine learning model can be trained to recognize patterns in the feature set that lead to a target variable value. A machine learning model that is trained to predict a target variable value may be referred to as a supervised learning model.

In some implementations, the machine learning model may be trained on a set of observations that do not include a target variable. This may be referred to as an unsupervised learning model. In this case, the machine learning model may learn patterns from the set of observations without labeling or supervision, and may provide output that indicates such patterns, such as by using clustering and/or association to identify related groups of items within the set of observations.

As shown by reference number 320, the machine learning system may train a machine learning model using the set of observations and using one or more machine learning algorithms, such as a regression algorithm, a decision tree algorithm, a neural network algorithm, a k-nearest neighbor algorithm, a support vector machine algorithm, and/or the like. After training, the machine learning system may store the machine learning model as a trained machine learning model 325 to be used to analyze new observations.

As shown by reference number 330, the machine learning system may apply the trained machine learning model 325 to a new observation, such as by receiving a new observation and inputting the new observation to the trained machine learning model 325. As shown, the new observation may include a first feature of current progress data X, a second feature of health parameters Y, a third feature of physiological parameters Z, and so on, as an example. The machine learning system may apply the trained machine learning model 325 to the new observation to generate an output (e.g., a result). The type of output may depend on the type of machine learning model and/or the type of machine learning task being performed. For example, the output may include a predicted value of a target variable, such as when supervised learning is employed. Additionally, or alternatively, the output may include information that identifies a cluster to which the new observation belongs, information that indicates a degree of similarity between the new observation and one or more other observations, and/or the like, such as when unsupervised learning is employed.

As an example, the trained machine learning model 325 may predict a value of movements A for the target variable of the movements for the new observation, as shown by reference number 335. Based on this prediction, the machine learning system may provide a first recommendation, may provide output for determination of a first recommendation, may perform a first automated action, may cause a first automated action to be performed (e.g., by instructing another device to perform the automated action), and/or the like.

In some implementations, the trained machine learning model 325 may classify (e.g., cluster) the new observation in a cluster, as shown by reference number 340. The observations within a cluster may have a threshold degree of similarity. As an example, if the machine learning system classifies the new observation in a first cluster (e.g., a current process data cluster), then the machine learning system may provide a first recommendation. Additionally, or alternatively, the machine learning system may perform a first automated action and/or may cause a first automated action to be performed (e.g., by instructing another device to perform the automated action) based on classifying the new observation in the first cluster.

As another example, if the machine learning system were to classify the new observation in a second cluster (e.g., a health parameters cluster), then the machine learning system may provide a second (e.g., different) recommendation and/or may perform or cause performance of a second (e.g., different) automated action.

In some implementations, the recommendation and/or the automated action associated with the new observation may be based on a target variable value having a particular label (e.g., classification, categorization, and/or the like), may be based on whether a target variable value satisfies one or more thresholds (e.g., whether the target variable value is greater than a threshold, is less than a threshold, is equal to a threshold, falls within a range of threshold values, and/or the like), may be based on a cluster in which the new observation is classified, and/or the like.

In this way, the machine learning system may apply a rigorous and automated process to determine movements for the personalized three-dimensional avatar. The machine learning system enables recognition and/or identification of tens, hundreds, thousands, or millions of features and/or feature values for tens, hundreds, thousands, or millions of observations, thereby increasing accuracy and consistency and reducing delay associated with determining movements for the personalized three-dimensional avatar relative to requiring computing resources to be allocated for tens, hundreds, or thousands of operators to manually determine movements for the personalized three-dimensional avatar.

As indicated above, FIG. 3 is provided as an example. Other examples may differ from what is described in connection with FIG. 3.

FIG. 4 is a flowchart of an example process 400 associated with generating personalized three-dimensional avatars for patient rehabilitation. One or more process blocks of FIG. 4 are performed by a computer system (e.g., computer 102 and/or monitoring system 210) and/or by another device or a group of devices separate from or including the computer system. Additionally, or alternatively, one or more process blocks of FIG. 4 may be performed by one or more components of computer 102, such as processor set 114, communication fabric 116, volatile memory 118, and/or monitoring code 150.

As shown in FIG. 4, process 400 may include receiving historical patient data that includes one or more of a wearable device feed, a video recording of a rehabilitation session, or a medical report associated with a patient (block 410). For example, the computer system may receive historical patient data that includes one or more of a wearable device feed, a video recording of a rehabilitation session, or a medical report associated with a patient, as described above. In some implementations, the historical patient data includes data identifying one or more of a joint angle, a pressure point, or a gait pattern of the patient. In some implementations, the historical patient data includes current progress data associated with the patient, one or more health parameters associated with the patient, and one or more physiological parameters associated with the patient.

As further shown in FIG. 4, process 400 may include anonymizing the historical patient data to generate historical anonymized patient data (block 420). For example, the computer system may anonymize the historical patient data to generate historical anonymized patient data, as described above.

As further shown in FIG. 4, process 400 may include creating a personalized three-dimensional avatar based on the historical anonymized patient data (block 430). For example, the computer system may create a personalized three-dimensional avatar based on the historical anonymized patient data, as described above. In some implementations, creating the personalized three-dimensional avatar based on the historical anonymized patient data includes processing the historical anonymized patient data, with a convolutional neural network model or a long short-term memory model, to create the personalized three-dimensional avatar.

As further shown in FIG. 4, process 400 may include processing the historical anonymized patient data, with a machine learning model, to predict one or more movements for rehabilitation of the patient (block 440). For example, the computer system may process the historical anonymized patient data, with a machine learning model, to predict one or more movements for rehabilitation of the patient, as described above.

As further shown in FIG. 4, process 400 may include updating the personalized three-dimensional avatar based on the one or more movements to generate an animated avatar performing the one or more movements (block 450). For example, the computer system may update the personalized three-dimensional avatar based on the one or more movements to generate an animated avatar performing the one or more movements, as described above.

As further shown in FIG. 4, process 400 may include providing the animated avatar to the patient in real-time through an augmented reality device (block 460). For example, the computer system may provide the animated avatar to the patient in real-time through an augmented reality device, as described above.

In some implementations, process 400 includes providing the animated avatar to a medical professional for review, receiving a modification to the one or more movements from the medical professional, and modifying the animated avatar based on the modification prior to providing the animated avatar to the patient. In some implementations, modifying the animated avatar based on the modification includes utilizing a generative adversarial network model to modify the animated avatar based on the modification.

In some implementations, process 400 includes receiving feedback from the patient during performance of the rehabilitation provided by the animated avatar, and adjusting the one or more movements of the animated avatar based on the feedback. In some implementations, process 400 includes generating a knowledge corpus based on the historical anonymized patient data. In some implementations, process 400 includes analyzing the historical patient data to identify deviations from a predetermined movement pattern.

In some implementations, process 400 includes identifying an environmental condition associated with the patient, and adjusting the one or more movements of the animated avatar based on the environmental condition. In some implementations, process 400 includes providing the animated avatar to a medical professional for review, receiving a modification to the one or more movements from the medical professional, and modifying the animated avatar based on the modification prior to providing the animated avatar to the patient. In some implementations, receiving the modification to the one or more movements from the medical professional includes receiving the modification to the one or more movements based on one or more of an interaction of the medical professional with the animated avatar, a demonstration of the modification to the one or more movements by the medical professional, or a gesture of the medical professional. In some implementations, process 400 includes receiving feedback from the patient during performance of the rehabilitation provided by the animated avatar, generating a summary of progress of the patient based on the feedback, and providing the summary of the progress to the patient and the medical professional.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

The foregoing disclosure provides illustration and description, but is not intended to be exhaustive or to limit the implementations to the precise forms disclosed. Modifications may be made in light of the above disclosure or may be acquired from practice of the implementations. For example, various implementations of this disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

A computer program product embodiment (“CPP embodiment” or “CPP”) is a term used in this disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, RAM, ROM, erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits / lands formed in a major surface of a disc), or any suitable combination of the foregoing. A computer-readable storage medium, as that term is used in this disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code - it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

When “a processor” or “one or more processors” (or another device or component, such as “a controller” or “one or more controllers”) is described or claimed (within a single claim or across multiple claims) as performing multiple operations or being configured to perform multiple operations, this language is intended to broadly cover a variety of processor architectures and environments. For example, unless explicitly claimed otherwise (e.g., via the use of “first processor” and “second processor” or other language that differentiates processors in the claims), this language is intended to cover a single processor performing or being configured to perform all of the operations, a group of processors collectively performing or being configured to perform all of the operations, a first processor performing or being configured to perform a first operation and a second processor performing or being configured to perform a second operation, or any combination of processors performing or being configured to perform the operations. For example, when a claim has the form “one or more processors configured to: perform X; perform Y; and perform Z,” that claim should be interpreted to mean “one or more processors configured to perform X; one or more (possibly different) processors configured to perform Y; and one or more (also possibly different) processors configured to perform Z.”

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method comprising:
receiving historical patient data that includes one or more of a wearable device feed, a video recording of a rehabilitation session, or a medical report associated with a patient;
anonymizing the historical patient data to generate historical anonymized patient data;
creating a personalized three-dimensional avatar based on the historical anonymized patient data;
processing the historical anonymized patient data, with a machine learning model, to predict one or more movements for rehabilitation of the patient;
updating the personalized three-dimensional avatar based on the one or more movements to generate an animated avatar performing the one or more movements; and
providing the animated avatar to the patient in real-time through an augmented reality device.

2. The method of claim 1, further comprising:
providing the animated avatar to a medical professional for review;
receiving a modification to the one or more movements from the medical professional; and
modifying the animated avatar based on the modification prior to providing the animated avatar to the patient.

3. The method of claim 2, wherein modifying the animated avatar based on the modification comprises:
utilizing a generative adversarial network model to modify the animated avatar based on the modification.

4. The method of claim 1, further comprising:
receiving feedback from the patient during performance of the rehabilitation provided by the animated avatar; and
adjusting the one or more movements of the animated avatar based on the feedback.

5. The method of claim 1, further comprising:
generating a knowledge corpus based on the historical anonymized patient data.

6. The method of claim 1, further comprising:
analyzing the historical patient data to identify deviations from a predetermined movement pattern.

7. The method of claim 1, wherein the historical patient data includes data identifying one or more of a joint angle, a pressure point, or a gait pattern of the patient.

8. A computer system, comprising:
a processor set;
one or more computer-readable storage media; and
program instructions stored on the one or more computer-readable storage media to cause the processor set to perform operations comprising:
receiving historical patient data that includes one or more of a wearable device feed, a video recording of a rehabilitation session, or a medical report associated with a patient;
anonymizing the historical patient data to generate historical anonymized patient data;
generating a knowledge corpus based on the historical anonymized patient data;
creating a personalized three-dimensional avatar based on the historical anonymized patient data;
processing the historical anonymized patient data, with a machine learning model, to predict one or more movements for rehabilitation of the patient;
updating the personalized three-dimensional avatar based on the one or more movements to generate an animated avatar performing the one or more movements; and
providing the animated avatar to the patient in real-time through an augmented reality device.

9. The computer system of claim 8, wherein creating the personalized three-dimensional avatar based on the historical anonymized patient data comprises:
processing the historical anonymized patient data, with a convolutional neural network model or a long short-term memory model, to create the personalized three-dimensional avatar.

10. The computer system of claim 8, wherein the operations further comprise:
identifying an environmental condition associated with the patient; and
adjusting the one or more movements of the animated avatar based on the environmental condition.

11. The computer system of claim 8, wherein the operations further comprise:
providing the animated avatar to a medical professional for review;
receiving a modification to the one or more movements from the medical professional; and
modifying the animated avatar based on the modification prior to providing the animated avatar to the patient.

12. The computer system of claim 11, wherein receiving the modification to the one or more movements from the medical professional comprises:
receiving the modification to the one or more movements based on one or more of an interaction of the medical professional with the animated avatar, a demonstration of the modification to the one or more movements by the medical professional, or a gesture of the medical professional.

13. The computer system of claim 8, wherein the historical patient data includes current progress data associated with the patient, one or more health parameters associated with the patient, and one or more physiological parameters associated with the patient.

14. The computer system of claim 8, wherein the operations further comprise:
receiving feedback from the patient during performance of the rehabilitation provided by the animated avatar;
generating a summary of progress of the patient based on the feedback; and
providing the summary of the progress to the patient and the medical professional.

15. A computer program product, comprising:
one or more computer-readable storage media; and
program instructions stored on the one or more computer-readable storage media to perform operations comprising:
receiving historical patient data that includes one or more of a wearable device feed, a video recording of a rehabilitation session, or a medical report associated with a patient;
anonymizing the historical patient data to generate historical anonymized patient data;
creating a personalized three-dimensional avatar based on the historical anonymized patient data;
processing the historical anonymized patient data, with a machine learning model, to predict one or more movements for rehabilitation of the patient;
updating the personalized three-dimensional avatar based on the one or more movements to generate an animated avatar performing the one or more movements;
providing the animated avatar to the patient in real-time through an augmented reality device;
receiving feedback from the patient during performance of the rehabilitation provided by the animated avatar; and
adjusting the one or more movements of the animated avatar based on the feedback.

16. The computer program product of claim 15, wherein the operations further comprise:
generating a knowledge corpus based on the historical anonymized patient data.

17. The computer program product of claim 15, wherein the operations further comprise:
providing the animated avatar to a medical professional for review;
receiving a modification to the one or more movements from the medical professional; and
modifying the animated avatar based on the modification prior to providing the animated avatar to the patient.

18. The computer program product of claim 15, wherein the historical patient data includes current progress data associated with the patient, one or more health parameters associated with the patient, and one or more physiological parameters associated with the patient.

19. The computer program product of claim 15, wherein creating the personalized three-dimensional avatar based on the historical anonymized patient data comprises:
processing the historical anonymized patient data, with a convolutional neural network model or a long short-term memory model, to create the personalized three-dimensional avatar.

20. The computer program product of claim 15, wherein the operations further comprise:
receiving feedback from the patient during performance of the rehabilitation provided by the animated avatar;
generating a summary of progress of the patient based on the feedback; and
providing the summary of the progress to the patient and the medical professional.
